(19) United States
(12) Patent Application Publication
(10) Pub. No.: US 2020/0297705 A1
(43) Pub. Date: Sep. 24, 2020

(54) ORAL LIQUID FORMULATIONS OF TACROLIMUS AND USES THEREOF

(71) Applicant: Gemini Laboratories, Inc., Bridgewater, NJ (US)

(72) Inventors: Hardik Patel, Hillsborough, NJ (US); Amitkumar Patel, Edison, NJ (US); Suhaskumar Patel, Edison, NJ (US)

(73) Assignee: Gemini Laboratories, Inc., Bridgewater, NJ (US)

(21) Appl. No.: 16/813,813

(22) Filed: Mar. 10, 2020

Related U.S. Application Data
(60) Provisional application No. 62/820,732, filed on Mar. 19, 2019.

(51) Int. Cl. A61K 31/439; A61K 9/00

(52) U.S. Cl. CPC A61K 31/439; A61K 9/0053

(57) ABSTRACT

The present disclosure relates to oral liquid tacrolimus formulations and the use in treating organ or stem cell transplant recipients or T-cell mediated diseases.

This application claims the benefit of U.S. Provisional Patent Application No. 62/820,732 filed Mar. 19, 2019, which is hereby incorporated herein by reference in its entirety.

### TECHNICAL FIELD

The present invention relates to oral liquid formulations of tacrolimus and method of using the oral liquid formulations to treat organ or stem cell transplant recipients or T-cell mediated diseases.

### BACKGROUND

Tacrolimus (also known as FK-506 or Fujimycin) is an immunosuppressive drug that can be used after organ or stem cell transplants to reduce the activity of the patient's immune system, and so lower the risk of organ rejection. It can reduce interleukin-2 (IL-2) production by T-cells. Tacrolimus can also be used in a topical preparation for the treatment of severe atopic dermatitis (eczema), severe refractory uveitis after bone marrow transplants, and the skin condition vitiligo. Tacrolimus is a 23-membered macrolide lactone discovered in 1987 from the fermentation broth of a Japanese soil sample that contained the bacteria Streptomyces tsukubaensis. The drug is sold under the trade names Prograf® given twice daily (oral) or as a continuous infusion (intravenous); Advagraf®, which is an extended release formulation allowing once daily dosing (oral); Envarsus,®, which is an extended release oral tablet with once daily dosing; and Protopic®, which is a topical formulation.

Tacrolimus has a poor solubility profile which presents challenges towards the development of a solution dosage form that could be administered to younger patients. Currently, there is no oral solution for Tacrolimus available on the market. For Prograf® the oral capsule options include 0.5, 1 and 5 mg dosages or intravenous administration. The U.S Food and Drug Administration recently approved an oral suspension form of Prograf® wherein the contents of unit dose packets containing solid granules comprising either 0.2 mg or 1 mg of Tacrolimus are suspended in water prior to administration. Because Tacrolimus has a narrow therapeutic index, it can be difficult to treat patients with the current oral dosage forms. Precise dosing is difficult to achieve with the current oral dosage forms and if more precise titration is required, the current dosage forms are intravenous. In addition, due to the complications of transplant operations, patients are oftentimes unable to swallow capsules. When this occurs, patients may be admitted to the hospital to receive Tacrolimus intravenously, which presents difficulties if there is no other reason for the patient to be admitted, or the patient may receive a compounded suspension from the pharmacy, which may offer inconsistent dosing if not shaken appropriately. The oral suspension also presents issues because of potential adverse mouth feel associated with the suspended particles and the possibility of inaccurate dosing if portions of the suspended Tacrolimus remain (adhered) to the cup in which the oral suspension is created.

### SUMMARY

The present invention is an oral liquid dosage formulation comprising: (a) tacrolimus; (b) one or more pharmaceutically acceptable solubilizers and (c) optionally one or more pharmaceutically acceptable excipients selected from the group consisting of flavoring agents, preservatives, buffering agents, pH adjusting agents, dispersants, carriers or combinations thereof.

In certain embodiments the oral liquid dosage formulation is: (i) a solution; (ii) non-aqueous or substantially water free; (iii) free or substantially free of any low molecular weight mono alcohols; (iv) free or substantially free of any dispersants such as a surfactant, wetting agent or emulsifying agent; and (v) any combination of (i), (ii), (iii) and (iv).

The present invention further includes a method for treating an organ or stem cell transplant recipient or a patient with a T-cell mediated disease comprising the step of orally administering the oral liquid dosage formulation to a patient in need of such treatment.

In yet another exemplary embodiment, the oral liquid dosage formulation may be used by measuring the appropriate volume of the oral liquid into a dosing device such as a cup, spoon or syringe without further manipulation such as suspending, diluting or combining with additional components. In a further embodiment of the ready to use oral liquid dosage formulation, the oral liquid may not require shaking, mixing, and/or stirring prior to use by the patient.

In yet another exemplary embodiment, the oral liquid dosage formulation can be stored for at least three months or longer under refrigerated conditions (i.e., between 0° C. and 10° C.). Alternatively, the oral liquid dosage formulation may be stored at ambient conditions or room temperature, i.e. without the need for refrigeration for three months or longer.

### DETAILED DESCRIPTION

The following detailed description is exemplary and explanatory and is intended to provide further explanation of the present disclosure described herein. Other advantages, and novel features will be readily apparent to one of ordinary skill in the art from the following detailed description of the present disclosure.

As used herein, the term “about” means a variability of 10% from the reference given, unless otherwise specified.

As used herein, the term “free” means the composition, formulation or material does not contain the component modified by the term “free”.

As used herein, the term “substantially free” means the composition, formulation or material contains small amounts of the component modified by the term “substantially free”. For examples a formulation that is substantially free of ethanol may contain less than 5.0%, 4.75%, 4.5%, 4.25%, 4.0%, 3.75%, 3.5%, 3.25%, 3.0%, 2.75%, 2.5%, 2.25%, 2.0%, 1.75%, 1.5%, 1.25%, 1.0%, 0.95%, 0.90%, 0.85%, 0.80%, 0.75%, 0.70%, 0.65%, 0.60%, 0.55%, 0.50%, 0.45%, 0.40%, 0.35%, 0.30%, 0.25%, 0.20%, 0.15%, or 0.10% weight percent ethanol.

As used herein the term “non-aqueous” should be accorded its normal meaning which may mean the composition, formulation or material does not contain any added or additional water other than the amount of water that commonly associated with the non-water components present in the composition, formulation or material. For example, a non-aqueous composition comprising solvents such as glycerin or propylene glycol will contain trace amounts of water because the United States Pharmacopeia (USP) allows glycerin to contain up to 5.0% water and propylene glycol to contain up to 0.2% water.

As used herein the term “low molecular weight” includes straight or branched C₁-C₁₂, preferably straight or branched C₁-C₈ and most preferably straight or branched C₁-C₆ chains.

A “patient” or “subject” is a mammal, e.g., a human or a veterinary patient or subject, e.g., mouse, rat, guinea pig, dog, cat, horse, cow, pig, or non-human primate, such as a monkey, chimpanzee, baboon or gorilla. In certain embodiments, the pharmaceutical formulations of the present disclosure can be administered to an adult patient or a pediatric patient. In one embodiment, the pediatric patient can be between the ages of six months to ten years.

The term “treating” or “treatment” is meant to encompass administering to a subject a compound of the present disclosure for the purposes of amelioration of one or more symptoms of a disease or disorder, including palliative care. As used herein, an “effective amount” or a “therapeutically effective amount” is used interchangeably and refers to an amount of a pharmaceutical formulation of the present disclosure which provides the desired treatment of a subject. As would be appreciated by one of ordinary skill in the art, the therapeutically effective amount of the present pharmaceutical formulations to treat a given disease, disorder or condition will vary from subject to subject, depending on factors such as age, general condition of the subject, the severity of the condition being treated, the particular compound and/or formulation administered, and the like. An appropriate therapeutically effective amount of the present pharmaceutical formulations suitable for any individual subject can be readily determined by one of ordinary skill in the art from the information provided herein.

The term “pharmaceutically acceptable” means suitable for use in humans or animals, for example as approved by a governmental regulatory agency (such as the US Food and Drug Administration) or listed in the USP or other generally recognized pharmacopeia, or which are generally recognized as safe (GRAS).

The oral liquid dosage formulations of the present invention comprises:
(a) tacrolimus;
(b) one or more pharmaceutically acceptable solubilizers; and
(c) optionally one or more pharmaceutically acceptable excipients selected from the group consisting of flavoring agents, preservatives, buffering agents, pH adjusting agents, dispersants, carriers or combinations thereof.

A list of potential solubilizers are provided on pages 3258 and 3261 of USP 29 (2006) which are incorporated herein by reference. Some examples of solubilizers may include in excipients such as emulsifying agents, surfactants, wetting agents, organic solvents, oils and combinations thereof. Examples of emulsifying agents, surfactants and wetting agents that maybe used as solubilizers are provided in greater detail below but some of the more preferred examples include but are not limited to polyoxyethylene sorbitan fatty acid esters, such as polysorbate 20, 40, 60 or 80, polyoxyethylene alkyl ethers, sorbitan esters, phosphatidylcholine, cyclodextrin and combinations thereof. Examples of organic solvents that may be used include but are not limited to low molecular weight mono alcohols such as ethanol; polyols, such as low molecular weight aliphatic triols, examples of which include glycerin and low molecular weight aliphatic diols, examples of which include propylene glycol; and polyethers, such as polyoxyalkylenes, examples of which include polyethylene glycols (“PEG”), preferably having a molecular weight less than 1000, less than 800 or less than 600. Examples of oils that may be used include, mineral oil, castor oil, vegetable and nut oils such as sunflower oil, corn oil, peanut oil, cottonseed oil, sesame oil, olive oil, canola oil, almond oil, safflower oil, soybean oil and combinations of the foregoing. Triacetin may also be used as a solubilizer in the dosage forms of the present invention. In certain embodiments, the solubilizer should be a liquid are room temperature. In certain embodiments, the solubilizer may be selected from the group consisting of polyethylene glycols, propylene glycol, glycerin, triacetin, ethanol, polysorbates, oils and combinations thereof.

The oral liquid dosage formulation may comprise from about 0.001 wt % to about 25 wt % of the tacrolimus, preferably about 0.01 wt % to about 20 wt % of the tacrolimus and most preferably about 0.05 wt % to about 15 wt % of the tacrolimus.

The oral liquid dosage formulation may comprise one or more pharmaceutically acceptable solubilizers. Preferably the oral liquid will comprise at least 0.5, 1, 5, 10, 15, 20, 25, 30, 35 40, 45, 50, 55, 60, 65, 70, 75, 80, 85, 90, or 95% w/v of one or more solubilizers as previously described. In certain embodiments, the total amount of one or more solubilizers employed in the oral liquid dosage formulations will comprise from about 0.5% w/v to about 99.999% w/v of the total weight of the liquid dosage formulation, about 1% w/v to about 99.99% w/v of the total weight of the liquid dosage formulation, about 5% w/v to about 99.999% w/v of the total weight of the liquid dosage formulation, preferably about 15% w/v to about 99.999% w/v of the total weight of the liquid dosage formulation and most preferably about 30% w/v to about 99.999% w/v of the total weight of the liquid dosage formulation.

In certain embodiments the oral liquid dosage formulation further comprise one or more pharmaceutically acceptable excipients selected from the group consisting of flavoring agents, preservatives, sweeteners, buffering agents, pH adjusting agents, dispersants, carriers or combinations thereof.

Examples of flavoring agents that may be employed in the liquid dosage form of the present invention include artificial sweeteners such as aspartame, sucralose, saccharin, dipotassium glycyrrhizinate, stevia, thaumatin, and flavorants such as citric acid, peppermint oil, wintergreen oil, menthol, lemon, lime, orange, grape, cherry, and vanilla extract and combinations of the foregoing. Additional flavoring agents are described in U.S. Pat. No. 6,027,746 which is incorporated herein by reference. The total amount of flavoring agent or agents will range from about 0.01 wt % to about 20 wt % of the liquid dosage formulation, preferably about 0.05 wt % to about 15 wt % of the liquid dosage formulation and most preferably about 0.01 wt % to about 10 wt % of the liquid dosage formulation.

Preservatives that may be used in the liquid dosage formulations include, antioxidants, antimicrobial agents, chelating agents and combinations thereof.

Examples of antioxidants that may be employed include ascorbic acid, ascorbyl palmitate, butylated hydroxyanisole, butylated hydroxytoluene, hypophosphorous acid, monothioglycerol, potassium metabisulfate, propyl gallate, sodium bisulfite, sodium formaldehyde sulfoxylate, sodium metabisulfate, sodium sulfate, sodium thiosulfate, sodium dioxide, tocopherol, and mixtures thereof. The antioxidant or antioxidants may be present in the liquid dosage formulations of the present invention in an amount from about 0.001 wt % to about 10 wt % of the liquid dosage formulation, preferably about 0.005 wt % to about 5 wt % of the liquid dosage formulation and most preferably about 0.01 wt % to about 2.5 wt % of the liquid dosage formulation.

Examples of antimicrobial agents that may be employed include benzalkonium chloride, benzethonium chloride, benzoic acid, benzyl alcohol, butylparaben, cetrimonium bromide, cetylpyridinium chloride, chlorobutanol, chlorocresol, ethylparaben, methylparaben, phenol, phenoxyethanol, phenylethyl alcohol, potassium benzoate, potassium sorbate, propylparaben, sodium benzoate, sodium dehydroacetate, sodium propionate, sorbic acid, thimersol, thymol and combinations thereof. The antimicrobial agent or agents may be present in the liquid dosage formulations of the present invention in an amount from about 0.001 wt % to about 5 wt % of the liquid dosage formulation, preferably about 0.005 wt % to about 2.5 wt % of the liquid dosage formulation and most preferably about 0.01 wt % to about 1.0 wt % of the liquid dosage formulation.

Examples of chelating agents that may be employed include citric acid and salts thereof, polyphosphates (e.g., sodium tripolyphosphate, hexametaphosphoric acid, sodium acid pyrophosphate, sodium pyrophosphate, tetra sodium pyrophosphate, sodium hexametaphosphate, sodium metaphosphate); aminocarboxylic acids (e.g., ethylenediaminetetraacetic acid (EDTA), 1,2-bis(2-amino-phenoxy)ethane-N,N,N′N′-tetraacetic acid (EGTA), ethylenebis(oxyethylenenitrilo)tetraacetic acid (BAPTA)) and salts thereof. The chelating agent or agents may be present in the liquid dosage formulations of the present invention in an amount from about 0.001 wt % to about 5 wt % of the liquid dosage formulation, preferably about 0.005 wt % to about 2.5 wt % of the liquid dosage formulation and most preferably about 0.01 wt % to about 1.0 wt % of the liquid dosage formulation.

Examples of buffering agents that may be employed include acetic acid, adipic acid, ammonium carbonate, ammonium phosphate, boric acid, citric acid, lactic acid, phosphoric acid, potassium citrate, potassium phosphate, sodium acetate, sodium citrate, sodium carbonate, potassium carbonate, sodium bicarbonate, potassium bicarbonate, sodium lactate, sodium phosphate, succinic acid, and combinations thereof. Typically the buffer will comprise a combination of the foregoing as to create a buffer system such as citric acid and sodium citrate or acetic acid and sodium acetate. The buffering agent or agents may be present in the liquid dosage formulations of the present invention in an amount from about 0.01 wt % to about 10 wt % of the liquid dosage formulation, preferably about 0.05 wt % to about 5 wt % of the liquid dosage formulation and most preferably about 0.1 wt % to about 2.5 wt % of the liquid dosage formulation.

Examples of pH adjusting agents that may be employed include, but are not limited to, any of the pharmaceutically acceptable acids or bases used to adjust the pH of pharmaceutical compositions. Examples of compounds typically used to adjust the pH of pharmaceutical compositions include hydrochloric acid, citric acid, lactic acid, tartaric acid, glacial acetic acid, sodium hydroxide, potassium hydroxide, arginine, lysine meglamine, triethanol amine, or combinations thereof. The amount of pH adjusting agents may be present in the liquid dosage formulations to obtain a desired pH typically from 3-8.

Dispersants that may be employed in the oral liquid formulations include but are not limited to surfactants, wetting agents and emulsifying agents. The surfactants further include non-ionic surfactants, ionic surfactants or a combination thereof. Examples of non-ionic surfactants include polyethoxylated castor oil, a polyoxyethylene alkyl ester, a polyglycolyzed glyceride, a sorbitan fatty acid ester, a glycerin fatty acid ester, a fatty acid polyglyceride, a fatty acid alcohol polyglycol ether, acetylene glycol, acetylene alcohol, an oxyalkylene block polymer, a polyoxyethylene alkyl ether, a polyoxyethylene alkylaryl ether, a polyoxyethylene styrylaryl ether, a polyoxyethylene glycol alkyl ether, a polyoxyethylene fatty acid ester, a polyoxyethylene sorbitan fatty acid ester, a polyoxyethylene glycerin fatty acid ester, a polyoxyethylene hydrogenated castor oil, a polyoxypropylene fatty acid ester, or a mixture of the foregoing. A further listing of possible non-ionic surfactants can be found on pages 1243-1249 of Martindale, The Extra Pharmacopoeia 29^{th} ed. which is incorporated herein by reference. Certain non-ionic surfactants include polyoxyethylene derivatives of polyol esters, such as Polysorbate 20 (TWEEN 20®), Polysorbate 40 (TWEEN 40®) Polysorbate 60 (TWEEN 60®), and Polysorbate 80 (TWEEN 80®), d-alpha tocopheryl polyethylene glycol 1000 succinate (TPGS), nonoxinols, poloxamers, sorbitan monolaurate, sorbitan monooleate, sorbitan monopalmitate, sorbitan monostearate, sorbitan sesquioleate, sorbitan trioleate, tyloxapol, and mixtures of the foregoing.

Ionic surfactants include, but are not limited to, carboxylates such as phospholipids, soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, phosphates, quaternary ammonium salts, and ethoxylated amines.

In certain embodiments, the dosage forms may further include a carrier. The carriers may include liquid excipients and diluents such as pharmaceutically acceptable solvents and co-solvents, preferably solvents and co-solvents that may be orally ingested. Examples of the liquid carriers have been described above and may in certain embodiments include or exclude water. The carriers may also include amounts of the foregoing solubilizers and dispersants, that exceed the amounts required to dissolve the tacrolimus.

In certain embodiments the oral liquid dosage formulations is solution, suspension or dispersion and most preferably a solution.

In certain embodiments the oral liquid dosage formulations is a non-aqueous solution, or a solution substantially free of water.

In certain embodiments the oral liquid dosage formulations is a solution that is free or substantially free any low molecular weight mono alcohols such as ethanol.

In certain embodiments the oral liquid dosage formulations is a solution free or substantially free of any surfactant.

In certain embodiments the oral liquid dosage formulations is a solution free or substantially free of a wetting agent.

In certain embodiments the oral liquid dosage formulations is a solution free or substantially free of an emulsifying agent.

In certain embodiments the oral liquid dosage formulations is a solution free or substantially free of a dispersant.

In certain embodiments the oral liquid dosage formulations is a solution comprising (a) tacrolimus; (b) one or more pharmaceutically acceptable solubilizers selected from the group consisting of polyethylene glycols, propylene glycol, glycerin, triacetin, oils and combinations thereof; (c) optionally one or more pharmaceutically acceptable excipients selected for the group consisting of flavoring agents, preservatives, buffering agents, pH adjusting agents, carriers or combinations thereof and wherein the oral liquid dosage formulation is non-aqueous, substantially free of low molecular weight mono alcohols such as ethanol, substantially free of any surfactant and substantially free of any emulsifier.

Suitable therapeutically effective amounts and dosage regimens utilizing the oral liquid formulations described herein can be selected by the ordinarily skilled clinician in accordance with a variety of factors, including species, age, weight, sex, and overall medical condition of the patient; the condition to be treated and its severity; and the renal and hepatic function of the patient.

For example the daily dose of the Tacrolimus administered from the oral liquid dosage formulations described herein will range from about 0.01 mg/kg/day to about 1 mg/kg/day with the total daily dose being administered in divided doses such as two, three or four times a day and preferably two administrations a day or a single administration every 12 hours. The following table provides some examples of the desired dosing:

| | |
|---|---|
| Adult Kidney Transplant Patients | 0.1-0.2 mg/kg/day administered in |
| | two doses every 12 hours |
| Adult Liver Transplant Patients | 0.1-0.15 mg/kg/day administered |
| | in two doses every 12 hours |
| Adult Heart Transplant Patients | 0.075 mg/kg/day administered in |
| | two doses every 12 hours |
| Pediatric Kidney Transplant Patients | 0.3 mg/kg/day administered in two |
| | doses every 12 hours |
| Pediatric Liver Transplant Patients | 0.15-0.2 mg/kg/day administered |
| | in two doses every 12 hours |
| Pediatric Heart Transplant Patients | 0.3 mg/kg/day administered in two |
| | doses every 12 hours |

The above doses may be adjusted based on other conditions such as the patient's renal or hepatic impairment levels. The doses may also be used or adjusted to treat patients with small bowel, pancreas, lung, trachea, skin, cornea, bone marrow and limb transplants.

In further embodiments, oral liquid dosage formulations of the present invention may comprise Tacrolimus in a concentration of about 0.01 mg/ml to about 10 mg/ml. The concentration can be less than about 10 mg/ml, 9 mg/ml, 8 mg/ml, 7 mg/ml, 6 mg/ml, 5 mg/ml, 4 mg/ml, 3 mg/ml, 2 mg/ml, 1.0 mg/ml, 0.9 mg/ml, 0.8 mg/ml, 0.7 mg/ml, 0.6 mg/ml, 0.5 mg/ml, 0.4 mg/ml, 0.3 mg/ml, 0.2 mg/ml, 0.1 mg/ml, 0.09 mg/ml, 0.08 mg/ml, 0.07 mg/ml, 0.06 mg/ml, 0.05 mg/ml, 0.04 mg/ml, 0.03 mg/ml, 0.02 mg/ml or 0.01 mg/ml. In one embodiment, the Tacrolimus is provided in a concentration of about 0.1 to about 1.5 mg/ml, or about 0.5 to about 1.0 mg/ml. In an embodiment, the Tacrolimus is provided in a concentration of about 0.1 mg/ml, about 0.5 mg/ml or about 1.0 mg/ml.

The oral liquid dosage formulations disclosed herein may comprise oral formulations having improved solubility and/or stability profiles. The formulations orally administrated via a solution can be bioequivalent to formulations administered via oral capsules, or any other administration route. The formulations disclosed herein are stable under standard storage conditions or accelerated conditions. The total amount of tacrolimus impurities in the formulations should range from about 0.1% to about 3%, preferably about 0.1% to about 2.5% and most preferably about 0.1% to about 2%. The standard storage conditions may comprise a temperature of about 20 to 25° C. (i.e., room temperature) and no more than about 40% Relative Humidity (RH). In one embodiment, the formulations disclosed herein are stable at room temperature for 3 months or longer, 6 months or longer, 12 months or longer, 18 months or longer or 24 months or longer under standard storage conditions. The stability of a formulation according to the present disclosure can be determined, for example, by measuring the physical state of the formulation, including the viscosity and presence of any discoloration and chemical stability by measuring assay of API & related compound.

In some embodiments, the oral liquid formulations of the disclosure are stable when subject to predetermined conditions for predetermined times. For example, the oral liquid formulations described herein can be stored at various predetermined temperatures and relative humidities for defined or predetermined time periods, for example in an open or closed container. In some embodiments, formulations of the disclosure are stable upon storage at about 0, 2, 5, 8, 10, 15, 20, 25, 30, 37, 40 or 45 degrees Celsius and about 0%, 5%, 10%, 15%, 20%, 25%, 30%, 35%, 40%, 45%, 50%, 55%, 60%, 65%, 70%, 75%, 80%, 85%, 90%, 95% or 100% relative humidity for a period of at least about 0.5, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 20, 25, 30, 35, 40, 45, 48, 50, 51, 52, 53, 55 or 60 hours 1 week, 2 weeks, 3 weeks or 4 week; 1 month, 2 months, 3 months, 4 months, 5 months or 6 months. The total amount of tacrolimus impurities in the formulations should range from about 0.1% to about 3%, preferably about 0.1% to about 2.5% and most preferably about 0.1% to about 2% when stored under the above temperatures and humidities in a closed container.

In some embodiments, oral liquid formulations described herein are stable upon storage in an open or closed container at: about 30 degrees Celsius and about 90 percent relative humidity for a period of at least about 20 hours; about 40 degrees Celsius and about 60 percent relative humidity for a period of at least about one week, two weeks or three weeks; about 40 degrees Celsius and about 75 percent relative humidity for a period of at least about one week, two weeks or three weeks; about 25 degrees Celsius and about 60 percent relative humidity for a period of at least about one month; about 40 degrees Celsius and about 75 percent relative humidity for a period of at least one month; about 25 degrees Celsius and about 60 percent relative humidity for a period of at least about 3 months; or 5 degrees Celsius at any relative humidity for a period of at least about three months. In some embodiments, “storage in an open container” means that the container was opened twice a day for a given period of time, for example up to four weeks, but was otherwise left closed. The total amount of tacrolimus impurities in the formulations should range from about 0.1% to about 3%, preferably about 0.1% to about 2.5% and most preferably about 0.1% to about 2% when stored under the above temperatures and humidities in a closed container.

In some embodiments, the oral liquid formulations described herein are stable when subject to predetermined conditions for predetermined times. For example, oral liquid formulations described herein can be stored at various predetermined temperatures and relative humidities for defined or predetermined time periods, for example in an open or closed container. In some embodiments (see Table 1), formulations of the disclosure are stable upon storage at about 5° C.±3° C. for up to 12 months and no significant degradation was observed. The stability results are provided in Table 4. A composition (see Table 2) was also stored at Controlled Room Temperature (“CRT”) (25° C./60% RH) for up to 3 months, and it exhibited good stability whereby the assay value was greater than 99%. However, the extent of impurities was increased as compared to the samples stored at 5° C.±3° C. (see Table 5).

In some embodiments, and particularly the oral liquid dosage formulations described in paragraphs [0018]-[0040] above, can be stored for at least three months, four months, five months, six months, or longer under refrigerated conditions, preferably at a temperature between about 0° C. and about 10° C. and more preferably between about 2° C. and about 8° C. when packaged in a single or multiple dose amber glass bottle with a conventional child resistant screw cap and with or without a press-in bottle adapter/orifice reducer with a dip tube. In certain embodiments the oral liquid dosage formulations will exhibit the following impurity profiles as determined by High Performance Liquid Chromatography analysis when stored under refrigerated conditions in an amber glass bottle with a conventional child resistant screw cap:

| | | | Most Preferred |
|---|---|---|---|
| | Amount | Preferred Amount | Amount |
| Tacrolimus | Less than 0.5% | Less than 0.25% | Less than 0.15% |
| 8-Epimer | | | |
| Tacrolimus Diene | Less than 0.5% | Less than 0.25% | Less than 0.15% |
| Total Known | Less than 1% | Less than 0.75% | Less than 0.5% |
| Impurities | | | |
| Individual | Less than 0.5% | Less than 0.4% | Less than 0.3% |
| Unknown | | | |
| Impurity | | | |
| Total Unknown | Less than 1% | Less than 0.75% | Less than 0.5% |
| Impurities | | | |

In some embodiments, and particularly the oral liquid dosage formulations described in paragraphs [0018]-[0040] above, can be stored for at least three months, four months, five months, six months, or longer at ambient conditions or room temperature, i.e. without the need for refrigeration, when packaged in a single or multiple dose amber glass bottle with a conventional child resistant screw cap as described above. In certain embodiments the oral liquid dosage formulations will exhibit the following impurity profiles as determined by High Performance Liquid Chromatography analysis when stored at ambient conditions in an amber glass bottle with a conventional child resistant screw cap:

| | | | Most Preferred |
|---|---|---|---|
| | Amount | Preferred Amount | Amount |
| Tacrolimus | Less than 0.5% | Less than 0.25% | Less than 0.15% |
| 8-Epimer | | | |
| Tacrolimus Diene | Less than 0.5% | Less than 0.25% | Less than 0.15% |
| Total Known | Less than 1% | Less than 0.75% | Less than 0.5% |
| Impurities | | | |
| Individual | Less than 0.5% | Less than 0.4% | Less than 0.3% |
| Unknown | | | |
| Impurity | | | |
| Total Unknown | Less than 1% | Less than 0.75% | Less than 0.5% |
| Impurities | | | |

Any suitable package or containers can be used to hold and dispense oral liquid dosage formulations described herein. In one embodiment the oral liquid dosage form, preferably a ready to use dosage form, is packaged in any suitable container such as an amber glass bottle with a child resistant closure. The amber glass bottler may further comprise a press in bottle adapter/orifice reducer, with or without a dip tube that will allow a user to withdraw the appropriate amount of the oral liquid dosage form into a dosing syringe. The packaged formulations may further include printed instructions describing the use and administration of the dosage form as well as dosing materials such as one or more dosing cups or one or more oral dosing syringes, with or without graduated volume markings. In certain embodiments the dosing cup or oral dosing syringe would comprise indicia indicated the volume of the dosage form in the dosing cup or oral dosing syringe, preferably in increments of 0.1 mL, 0.25 mL, 0.5 mL or combinations thereof. The packages can be a single dose package or can contain multiple doses of the oral liquid dosage formulation.

The present invention also includes a method for manufacturing oral liquid dosage formulations, and particularly the oral liquid dosage formulations described in paragraphs [0018]-[0040] above, comprising: adding one or more pharmaceutically acceptable solubilizers to a clean manufacturing tank and sparging and/or purging with one or more inert gases, for example nitrogen, adding the Tacrolimus and any additional pharmaceutically acceptable excipients such as flavoring agents, sweeteners, preservatives, buffering agents or pH adjusting agents to the manufacturing tank with continuous sparging and/or purging with an inert gas (e.g., nitrogen). Additional organic solubilizers may be added to obtain the desired final volume and the oral liquid dosage formulation, preferably a solution, can be filled into single or multiple dose de-ionized amber glass bottles with continuous nitrogen sparging and/or purging and sealed with a screw cap.

In certain embodiments the single or multiple dose amber glass bottles with the oral liquid dosage formulation, and particularly the oral liquid dosage formulations described in paragraphs [0018]-[0040] above, are “ready to use”. Specifically, the patient or care provider will measure the appropriate volume of the oral liquid into a dosing device such as a cup, spoon or syringe without further manipulation of the oral liquid dosage formulation such as suspending, diluting or combining with additional components. Once the appropriate amount volume of the oral liquid dosage formulation is in the dosing device, the patient or care provider may administer the oral liquid dosage formulation. In the case of a single dose bottle, the patient or care provider will not be required to measure the appropriate volume into a dosing device but may orally administer the oral liquid dosage formulation directly from the bottle to the patient. In certain embodiments, the oral liquid dosage form will not require any shaking, mixing or stirring prior to use or administration to a patient.

### EXAMPLES

Below are examples of formulations of Tacrolimus oral solutions according to embodiments of the present disclosure. Comparative assessments of the bioavailability and pharmacokinetic parameters of Tacrolimus in solution dosage forms according to the present disclosure and that of capsule dosage forms can be conducted.

### Example 1

### Formulation Composition of Tacrolimus Oral Solution, 0.5 mg/mL & 1.0 mg/mL

TABLE 1

| | 0.5 mg/mL | 1.0 mg/mL | |
|---|---|---|---|
| | strength | strength | |
| Ingredients | % w/v | % w/v | Function |
| Tacrolimus | 0.05 | 0.1 | Active |
| Polysorbate 80, NF | 0.8 | 0.8 | Dispersant |
| Phosal 50 PG² | 99.65 | 99.60 | Solubilizer |
| Nitrogen, NF | QS | QS | Sparging Agent |

^{1.} Density of the product is approximately 1.005 g/mL.
² For composition of Phosal 50 PG, please see Table 6 below.
QS = Quantity Sufficient.

The compositions of Example 1 were prepared under yellow light throughout complete manufacturing according to the following procedure.
1. The Phosal 50 PG was added to a clean manufacturing tank and stirred using a propeller mixer.
2. The Phosal 50 PG was sparged for at least 10 minutes with Nitrogen NF supplied from a Nitrogen Cylinder with the tip of a nitrogen supply tube dipped into the Phosal 50 PG.
3. Polysorbate 80, NF was added to the manufacturing tank of Step #2 and the Polysorbate 80 container was rinsed with Phosal 50 PG and the rinsate transferred to the manufacturing tank. The resulting composition was mixed for 20 minutes with continuous Nitrogen sparging/purging.
4. Tacrolimus was screened through an 80 mesh stainless steel screen and added to the manufacturing tank of Step #3 and mixed for 10 minutes with continuous Nitrogen sparging/purging. After 10 minutes the propeller mixer was stopped, the lid of the manufacturing tank opened and the side walls scrapped with a stainless steel scrapper. After scrapping the lid was closed, the propeller mixer was turned “ON” and the composition was mixed further for 170 minutes with continuous Nitrogen sparging/purging to achieve a clear solution.
5. The solution from manufacturing tank of Step #4 is transferred to a holding tank through a 100 mesh stainless steel screen followed by industrial metal detector using a transfer pump using Platinum cured silicon tubing.
6. The propeller mixer of the holding tank was turned “ON” and solution was mixed for 20 minutes with continuous Nitrogen sparging/purging.
7. In-process samples were taken.
8. After the in-process samples were taken, the lid of the holding tank was tightly closed and the solution was continuously sparged/purged with Nitrogen until the bulk solution was filled and packaged.
9. Filling and Packaging Process:
9a. The holding tank was connected to the filling machine using flexible tubing (Platinum cured silicone) and the bulk solution was continuously sparged/purged with Nitrogen.
9b. The IPS Filling Machine was set to the required fill weight (2 oz per bottle).
9c. The Nitrogen supply from the cylinder should be set to also provide Nitrogen for one of the filling Nozzle of the Filling Machine.
9d. Amber glass bottles that had been de-ionized with air were filled the set amount of solution, approximately 2 oz. per bottle (Target Fill Weight).
9e. Once the bottle was filled to the Target Fill Weight it tightly the cap, using a Kaps All Capper.

### Example 2

### Formulation Composition of Tacrolimus Oral Solution, 0.5 mg/mL & 1.0 mg/mL

TABLE 2

| | 0.5 mg/mL | 1.0 mg/mL | |
|---|---|---|---|
| | strength | strength | |
| Ingredients | % w/v | % w/v | Function |
| Tacrolimus | 0.05 | 0.1 | Active |
| Polysorbate 80, NF | 1.0 | 1.0 | Dispersant |
| Phosal 50 PG² | 99.45 | 99.40 | Solubilizer |
| Nitrogen, NF | QS | QS | Sparging Agent |

^{1.} Density of the product is approximately 1.005 g/mL.
² For composition of Phosal 50 PG, please see Table 6 below.
QS = Quantity Sufficient.

The compositions of Example 2 were manufactured and packaged similar to the procedure described in Example 1.

### Example 3

### Formulation Composition of Tacrolimus Oral Solution, 0.5 mg/mL & 1.0 mg/mL

TABLE 3

| | 0.5 mg/mL | 1.0 mg/mL | |
|---|---|---|---|
| | strength | strength | |
| Ingredients | % w/v | % w/v | Function |
| Tacrolimus | 0.05 | 0.1 | Active |
| Polysorbate 80, NF | 1.2 | 1.2 | Dispersant |
| Phosal 50 PG² | 99.25 | 99.20 | Solubilizer |
| Nitrogen, NF | QS | QS | Sparging Agent |

^{1.} Density of the product is approximately 1.005 g/mL.
² For composition of Phosal 50 PG, please see Table 6 below.
QS = Quantity Sufficient.

The compositions of Example 3 were manufactured and packaged similar to the procedure described in Example 1.

Tables 4 and 5 present stability characteristics of the compositions provided in Examples 1 & 2 above.

TABLE 4
Example 1
Tacrolimus Oral Solution, 1 mg/mL
(Related Compound and Assay Data)
Related Compound

| | Tacrolimus Oral Solution, |
|---|---|
| | 1 mg/mL (Example: 1) |
| | (2-8)° C./12 Months |
| Impurity Name | % Impurity |
| Tacrolimus 8-Epimer | 0.047 |
| Tacrolimus Related | 0.012 |
| Compound A | |
| Tacrolimus 8-Propyl analog | ND |
| Tacrolimus Diene | 0.013 |
| Unknown-1 | 0.008@ RRT 1.25 |
| % Total Impurity | 0.080 |

Assay

| | | Example: 1 | |
|---|---|---|---|
| | Example: 1 | (1.0 mg/mL | Example: 1 |
| | (1.0 mg/mL | strength) 25° | (1.0 mg/mL |
| | strength) (2-8)° | C./60% RH/ | strength) (2-8)° |
| | C./4 Months | 4 Months | C./12 Months |
| | (% Assay) | (% Assay) | (% Assay) |
| % Assay | 102.9 | 100.8 | 99.5 |

TABLE 5
Example 2
Tacrolimus Oral Solution, 1 mg/mL
(Related Compound and Assay Data)
Related Compound

| | | | (Example 2; |
|---|---|---|---|
| | (Example 2; | (Example 2; | 1 mg/2 mL |
| | 1 mg/2 mL | 1 mg/2 mL | Strength) 25° |
| | Strength) | Strength) (2-8)° | C./60% RH/ |
| | Initial | C./3 Months | 3 Months |
| Impurity Name | % Impurity | % Impurity | % Impurity |
| Tacrolimus 8- | 0.052 | 0.087 | 0.267 |
| Epimer | | | |
| Tacrolimus | ND | 0.018 | 0.018 |
| Related | | | |
| Compound A | | | |
| Tacrolimus 8- | ND | ND | ND |
| Propyl analog | | | |
| Tacrolimus Diene | — | 0.006 | 0.027 |
| Unknown-1 | ND@ | 0.011@ | 0.062@ |
| | RRT 0.93 | RRT 1.25 | RRT 1.25 |
| % Total Impurity | 0.052 | 0.122 | 0.374 |

Assay

| | | | (Example 2; |
|---|---|---|---|
| | (Example 2; | (Example 2; | 1 mg/2 mL |
| | 1 mg/2 mL | 1 mg/2 mL | Strength) 25° |
| | Strength) | Strength) (2-8)° | C./60% RH/ |
| | Initial | C./3 Months | 3 Months |
| | (% Assay) | (% Assay) | (% Assay) |
| % Assay | 101.7 | 100.5 | 99.5 |

TABLE 6
PHOSAL ® 50 PG

| Ingredient | Content |
|---|---|
| Phosphatidylcholine | ≥50% |
| Propylene Glycol | 25-50% |
| Sunflower Seed Oil Glyceride | 1-5% |
| Soy Acid | 1-5% |
| Alcohol | 1-5% |
| Ascorbyl Palmitate | 0.1-1% |
| Tocopherol | 0.1-1% |

### Example 4

The following table exemplifies an oral liquid Tacrolimus dosage form that was prepared by a procedure similar to that described in Example 1:

| Ingredients | % w/v |
|---|---|
| Tacrolimus | 0.1% |
| Propylene Glycol | 40.0% |
| Purified water | 20% |
| Cyclodextrin | 20.0% |
| Sodium Saccharin | 0.05% |
| Glycerin | 10.0% |
| Methylparaben | 0.1% |
| Propylparaben | 0.02% |
| Purified Water | QS to 100% |

The following Tables 8-9 show purity of a Tacrolimus oral solution of 1 mg/ml as a percent assay, based on the batch descriptions in Table 7.

TABLE 7
Batch Description

| Batch # | Batch Composition | Batch Size |
|---|---|---|
| Example 3 (RD6185-05) | Batch with Phosal 50 PG | 2.0 Liter |
| Example 4 (RD6185-07) | Batch with Cyclodextrin | 2.0 Liter |

TABLE 8
Assay for Example 3 (RD6185-05)[Batch with Phosal 50 PG]

| Batch # | % Assay |
|---|---|
| API_Lot # DMP (079)-38 | 97.1% |
| RD6185-05_CRT (3 Month) | 92.5% |
| RD6185-05_(2-8 C.) (3 Month) | 96.8% |

TABLE 9
Assay for Example 4 (RD6185-07)[Batch with Cyclodextrin]

| Batch # | % Assay |
|---|---|
| API_Lot # DMP (079)-38 | 97.1% |
| RD6185-07_CRT (Initial) | 89.9% |
| RD6185-07_(2-8 C.) (Initial) | 89.9% |

Tables 8-9 show the results of purity assays comparing the active pharmaceutical ingredient (API) with the batch compositions shown in Table 7 at room temperature (CRT) and refrigeration conditions (2-8° C.).

### Example 5

A Tacrolimus oral solution with the following composition was prepared:

| Ingredients | % w/v |
|---|---|
| Tacrolimus | 0.1% |
| Propylene Glycol | 30.0% |
| Saccharin Sodium | 0.2% |
| UB 1456 Orange Extract | 0.2% |
| Glycerin | QS to 100% |

The composition of Example 5 was prepared under yellow light throughout complete manufacturing according to the following procedure.
1. The Propylene Glycol was added to a clean manufacturing tank and stirred using a propeller mixer.
2. The Propylene Glycol was sparged for at least 10 minutes with Nitrogen NF supplied form a Nitrogen Cylinder with the tip of a nitrogen supply tube dipped into the Propylene Glycol.
3. The Tacrolimus was added to the manufacturing tank of Step #2 and the resulting composition was mixed for 15 minutes with continuous Nitrogen sparging/purging.
4. The Saccharin Sodium was added to the manufacturing tank of Step #3 and the resulting composition was mixed for 40 minutes with continuous Nitrogen sparging/purging.
5. The UB 1456 Orange Extract was added to the manufacturing tank of Step #4 and the resulting composition was mixed for 5 minutes with continuous Nitrogen sparging/purging
6. The solution from manufacturing tank of Step #5 was transferred to a holding tank through a 100 mesh stainless steel screen followed by industrial metal detector using a transfer pump using Platinum cured silicon tubing.
7. The propeller mixer of the holding tank was turned “ON” and solution was mixed for 20 minutes with continuous Nitrogen sparging/purging.
8. In-process samples were taken.
9. After the in-process samples were taken, the lid of the holding tank was tightly closed and the solution was continuously sparged/purged with Nitrogen until the bulk solution was filled and packaged.
10. Filling and Packaging Process:
10a. The holding tank was connected to the filling machine using flexible tubing (Platinum cured silicone) and the bulk solution was continuously sparged/purged with Nitrogen.
10b. The IPS Filling Machine was set to the required fill weight (2 oz per bottle).
10c. The Nitrogen supply from cylinder should be set to also provide Nitrogen for one of the filling Nozzle of the Filling Machine.
10d. Amber glass bottles that had been de-ionized with air were filled the set amount of solution, approximately 2 oz. per bottle (Target Fill Weight).
10e. Once the bottle was filled to the Target Fill Weight it tightly the cap, using a Kaps All Capper.

The composition of Example 5, packaged in 60 mL amber colored glass bottles with a tightly sealed child resistant closure, was placed on stability and showed the following stability profile:

### Related Compound:

| | Tacrolimus | Tacrolimus | Any | | | |
|---|---|---|---|---|---|---|
| | 8-Epimer | Diene | Unknown | Total | Total | |
| Stability | Limit: | Limit: | Imp | Known | Unknown | Total |
| Condition | 0.5% | 0.5% | Limit: 0.2% | Imp | Imp | Impurities |
| Initial | ND | ND | ND | ND | ND | ND |
| 2-8° C./3 m | ND | ND | ND | ND | ND | ND |
| 2-8° C./6 m | 0.038 | 0.062 | 0.033 | 0.1 | 0.03 | 0.13 |
| CRT/6 m | 1.032 | 1.08 | 0.264 | 2.112 | 0.5 | 2.612 |

*CRT = 25° C./60% relative humidity

### Assay Data:

| Stability Condition | Assay |
|---|---|
| Initial | 99.3 |
| 2-8° C./3 m | 102.7 |
| 2-8° C./6 m | 98.4 |
| CRT/6 m | 86.9 |

### Example 6

A Tacrolimus oral solution with the following composition was prepared:

| Ingredients | % w/v |
|---|---|
| Tacrolimus | 0.1% |
| Polyethylene Glycol 400 | 40.0% |
| Saccharin Sodium | 0.3% |
| UB 1456 Orange Extract | 0.2% |
| Propylene Glycol | QS to 100% |

The above solution was prepared and packaged according to the general procedure described in Example 5 with the following deviations:
1. Propylene glycol and polyethylene glycol 400 were initially added to manufacturing tank and mixed for 15 minutes with Nitrogen sparging/purging.
2. The Tacrolimus was added to the solution of step 1 and mixed for 30 minutes.
3. The saccharin sodium was added to the solution of step 2 and mixed for 40 minutes.
4. The UB 1456 Orange Extract was added to the solution of step 3 and mixed for 5 minutes.
5. Propylene Glycol was added to the solution of step 4 to make up the final volume and mixed for 10 minutes.

The invention illustratively described herein suitably may be practiced in the absence of any element or elements, limitation or limitations which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. An oral liquid dosage formulation comprising:
(a) tacrolimus;
(b) one or more pharmaceutically acceptable solubilizers; and
(c) optionally one or more pharmaceutically acceptable excipients selected from the group consisting of flavoring agents, sweeteners, preservatives, buffering agents, pH adjusting agents, dispersants, carriers or combinations thereof wherein the formulation is a non-aqueous solution.

2. The oral liquid dosage formulation of claim 1 wherein the one or more pharmaceutically acceptable solubilizers are selected from the group consisting of selected from the group consisting of polyethylene glycols, propylene glycol, glycerin, triacetin, ethanol, polysorbates, oils and combinations thereof.

3. The oral liquid dosage formulation of claim 2 wherein the one or more pharmaceutically acceptable solubilizers are selected from the group consisting of selected from the group consisting of polyethylene glycols, propylene glycol, glycerin, triacetin, oils and combinations thereof.

4. The oral liquid dosage formulation of claim 1 wherein the one or more pharmaceutically acceptable solubilizers are liquid at room temperature.

5. The oral liquid dosage formulation of claim 1 wherein the formulation is substantially free of a surfactant, wetting agent or emulsifying agent.

6. The oral liquid dosage formulation of claim 1 wherein the formulation is substantially free of low molecular weight mono alcohols.

7. An oral liquid dosage formulation comprising:
(a) tacrolimus;
(b) one or more pharmaceutically acceptable solubilizers selected from the group consisting of selected from the group consisting of polyethylene glycols, propylene glycol, glycerin, triacetin, ethanol, polysorbates, oils and combinations thereof; and
(c) optionally one or more pharmaceutically acceptable excipients selected from the group consisting of flavoring agents, sweeteners, preservatives, buffering agents, pH adjusting agents, or combinations thereof
wherein the formulation is a non-aqueous solution, free of a surfactant, wetting agent, emulsifying agent and low molecular weight mono alcohols.
